(19) United States
(12) Patent Application Publication
(10) Pub. No.: US 2021/0181203 A1
(43) Pub. Date: Jun. 17, 2021

(54) TEXTURED COMPOSITIONS, METHODS, AND SYSTEMS FOR RETAINING BIOMOLECULES

(71) Applicant: 3dbiosurfaces Technologies, LLC, Tucson, AZ (US)

(72) Inventors: Alan Gordon Goodyear, Tucson, AZ (US); Barry Beroth, Tucson, AZ (US)

(21) Appl. No.: 16/081,759

(22) PCT Filed: Mar. 9, 2017
(86) PCT No.: PCT/US17/21633

§ 371 Date: Aug. 31, 2018

Related U.S. Application Data
(60) Provisional application No. 62/305,679, filed on Mar. 9, 2016.

(51) Int. Cl. G01N 33/68; G01N 33/531

(52) U.S. Cl. CPC G01N 33/68; G01N 33/531

(57) ABSTRACT

A system featuring a textured surface having elements, such as a plurality of microfeatures and/or microstructures, that increase the surface area as compared to a surface without the elements, wherein a non-volatile solvent is disposed in at least a portion of the elements of the textured surface. The non-volatile solvent may be used for retaining biomolecules, such as but not limited to biomolecules that may benefit from an environment that protects conformational structure, for example proteins.

### CROSS REFERENCE

This application claims priority to U.S. Provisional Patent Application No. 62/305,679 filed Mar. 9, 2016, the specification(s) of which is/are incorporated herein in their entirety by reference.

### FIELD OF THE INVENTION

The present invention relates to systems with textured substrates, wherein biomolecules may be retained within said texture. The systems may feature a non-volatile solvent exhibiting negligible vapor pressure. Biomolecules may include but are not limited to those that may benefit from retention of a particular conformational structure.

### BACKGROUND OF THE INVENTION

Tethering proteins and other biomolecules that require shape and structure to function in assays (such as microarrays) has proved to be a difficult task in that proteins or other biomolecules may be damaged as a result of the method used to attach molecules to assay substrates (such as microarray substrates). Additionally, further damage to proteins or other biomolecules may occur as the solvent (e.g., water-based solvent) dries out. This latter problem is often dealt with by keeping the assay substrate (e.g., microarray substrate) contained within a suitable hydrating environment. However, this limits the ability for assay substrates (e.g., microarray substrates) that have been printed at one location to be transported to another distinct location. Several suppliers of microarrays have practiced the use of a porous layer, hydrogels (e.g., hydrogels that swell), polymer clusters, or polymer mesh. For example, EP 2,233,925 describes immobilizing biomolecules in a polymer surface modified substrate. EP 1,437,368 also describes methods for producing polymer carriers for retaining biomolecules. These methods and surfaces have had limited success (and porous surfaces also dry out).

It was surprisingly discovered that the use of a non-volatile solvent (e.g., solvent having little, negligible, or no vapor pressure, e.g., ionic liquid) on a textured surface helped to retain proteins and preserve the conformational structure of the proteins, thereby improving assay results. Without wishing to limit the present invention to any theory or mechanism, it is possible that the use of an ionic liquid may help repair structural damage that may have occurred to the protein or other biomolecule.

The present invention features a system comprising a textured substrate and a non-volatile solvent within at least portion of the textured substrate, wherein biomolecules may be retained within or by the textured surface in the non-volatile solvent. In some embodiments, the textured surface comprises microfeatures and/or microstructures that increase the surface area as compared to a flat surface (see flat surface of FIG. 1A). The present invention provides a substrate and a solvent that both retains the biomolecules (e.g., proteins or other biomolecules) and helps to keep the biomolecules in an effective hydrated condition. In some embodiments, no attachment or anchoring is necessarily required for the biomolecule (e.g., protein) in order for it to be used in an assay (e.g., a diagnostic test). The confinement of the biomolecules (e.g., proteins) within or by the microfeatures and/or microstructures may be sufficient to achieve both localization and retention. However, the present invention also includes the use of attachment chemistries, which are known to one of ordinary skill in the art.

The present invention, e.g., the textured surface featuring biomolecules in a non-volatile solvent (e.g., ionic liquid) retained within or by the microfeatures and/or microstructures of the textured surface, has an extended shelf life as compared to a flat surface with biomolecules anchored thereon because the biomolecules in the present invention are retained in an environment that is hydrated, that helps to preserve the conformational structure of the biomolecules, and that does not dry out. Thus, surfaces that incorporate the textured surfaces of the present invention allow for packaging, transporting, and storing of biomolecules adapted for assays such as microarrays or other applicable assays.

Any feature or element (e.g., microfeature or microstructure) or combination of features or elements (e.g., microfeatures and/or microstructures) described herein is included within the scope of the present invention.

### SUMMARY OF THE INVENTION

The present invention features a system comprising a textured surface with elements that provide an increase in surface area as compared to a non-textured surface. A non-volatile solvent exhibiting negligible vapor pressure is contained within at least an element (e.g., microstructure and/or microfeature) of the texture. A biomolecule may be solvated in the non-volatile solvent.

The present invention also features a method of preparing a system for retaining a biomolecule. In some embodiments, the method comprises introducing a non-volatile solvent exhibiting negligible vapor pressure to a textured surface of the present invention, wherein the biomolecule is solvated in the non-volatile solvent. The non-volatile solvent with the biomolecule is adsorbed into the elements of the textured surface for retention of the biomolecule within or by the elements of the textured surface.

The present invention also features a method of storing a biomolecule. In some embodiments, the method comprises solvating the biomolecule in a non-volatile solvent and introducing the biomolecule to a system comprising a textured surface of the present invention, wherein the non-volatile solvent with the biomolecule is adsorbed into the elements of the textured surface for retention of the biomolecule within or by the elements of the textured surface. The biomolecule may be stored for a period of time (e.g., at least 1 day, at least 1 week, at least 1 month, at least 6 months, at least 1 year, etc.).

The present invention also features a method of transporting a biomolecule. In some embodiments, the method comprises solvating the biomolecule in a non-volatile solvent and introducing the biomolecule to a system comprising a textured surface of the present invention, wherein the non-volatile solvent with the biomolecule is adsorbed into the elements of the textured surface for retention of the biomolecule within or by the elements of the textured surface. The system may be moved from one location to a different location.

The systems of the present invention may help protect the conformational structure of the biomolecule, e.g., the biomolecule may retain its conformational structure in the non-volatile solvent. The biomolecule may require a predetermined conformational structure to function for an assay. The system may help retain and prevent denaturation of the biomolecule (e.g., protein). The systems of the present invention may be used for a variety of purposes such as, but not limited to, protein detection or other assays.

As described herein, in some embodiments, the elements of the textured surface comprise microstructures. In some embodiments, the elements of the textured surface comprise microfeatures. In some embodiments, the elements of the textured surface comprise microstructures and microfeatures.

As described herein, in some embodiments, the system comprises a layer (e.g., gel, film, etc.) overlaying the non-volatile solvent such that the non-volatile solvent is retained in the elements of the textured surface.

As described herein, in some embodiments, the system features energetic moieties and/or polymerizable molecules. For example, a polymerizable molecule may be contained in the non-volatile solvent and/or element of the textured surface, e.g., the polymerizable molecule may be attached to an element of the textured surface.

Any feature (e.g., microfeature or microstructure) or combination of features (e.g., microfeatures and/or microstructures) described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows a flat surface (for purposes of comparison to a textured surface with microfeatures).

FIG. 1B and FIG. 10 show textured surfaces comprising microfeatures (110).

FIG. 1D shows a textured surface comprising microfeatures (110) with microstructures (120) in and between the microfeatures (110). Note the microfeatures can create cavities, e.g., with microstructures at the bottom and/or on the side of the cavity. Note that microstructures may be on the top of the microfeatures as well as on the side (and also in between the microfeatures).

FIG. 1E shows a textured surface comprising microstructures (120).

FIG. 2A shows a well (105) with microstructures (120) disposed on the bottom surface of the well. A well may refer to a well in a microarray substrate or any other appropriate well such as a well of a multi-well plate.

FIG. 2B shows a well (105) with both microfeatures (110) and microstructures (120) disposed on the bottom surface of the well.

FIG. 3A shows a detailed view of microstructures (120) (but may also represent microfeatures (110)).

FIG. 3B shows the microfeatures/microstructures (110, 120) of FIG. 3A and a non-volatile solvent disposed in the microfeatures/microstructures. Also shown are biomolecules (101), e.g., proteins, retained by a non-volatile solvent (102).

FIG. 3C shows the microfeatures/microstructures (110, 120) of FIG. 3A and a non-volatile solvent disposed in the microfeatures/microstructures. Also shown are biomolecules (101) and polymerizable molecules (103) added to the non-volatile solvent (102), wherein the polymerizable molecules have polymerized to form a network of polymers constraining the biomolecules (surrounding them but not attached to them).

### DETAILED DESCRIPTION OF THE INVENTION

A microarray is a general term for a typically smooth, flat surface of a substrate wherein a plurality of distinct and separate locations upon the surface of the substrate is established at the start of the analysis by populating each of the locations with probes (e.g. biomolecules) of a known composition. The probes are fixed to their unique locations on the flat substrate by an attachment layer that prevents detachment of the probes from the substrate. After a number of processing steps, the entire microarray is flooded with biomolecules of unknown composition (targets). When a target is captured by a bound probe, it may be possible infer the nature and composition of the target. The sensitivity of this process is limited by the number of probe-target complexes within each specific location on the microarray. In some cases, a microarray may feature a plurality of small wells.

A multi-well plate is a single plate-like device with a number of depressions (wells) in it surface, e.g., 96 wells, 384 wells, etc. Multi-well plates are used for a variety of parallel analyses, including but not limited to the process described for the microarray above.

As used herein, a biomolecule may refer to an oligonucleotide (e.g., DNA, RNA), protein or peptide (e.g., antibody, antigen), lipid, carbohydrate or other molecule found in biological entities (e.g., a peptide nucleic acid, a fatty acid, a vitamin, a cofactor, a purine, a pyrimidine, formysin, a phytochrome, a phyyofluor, or phycobiliprotein, etc.) or entire biological entities (e.g., a virus, a phage, a prion a bacteria, etc.) or cells (e.g., eukaryotic or prokaryotic cells, etc.). The present invention is not limited to the aforementioned biomolecules.

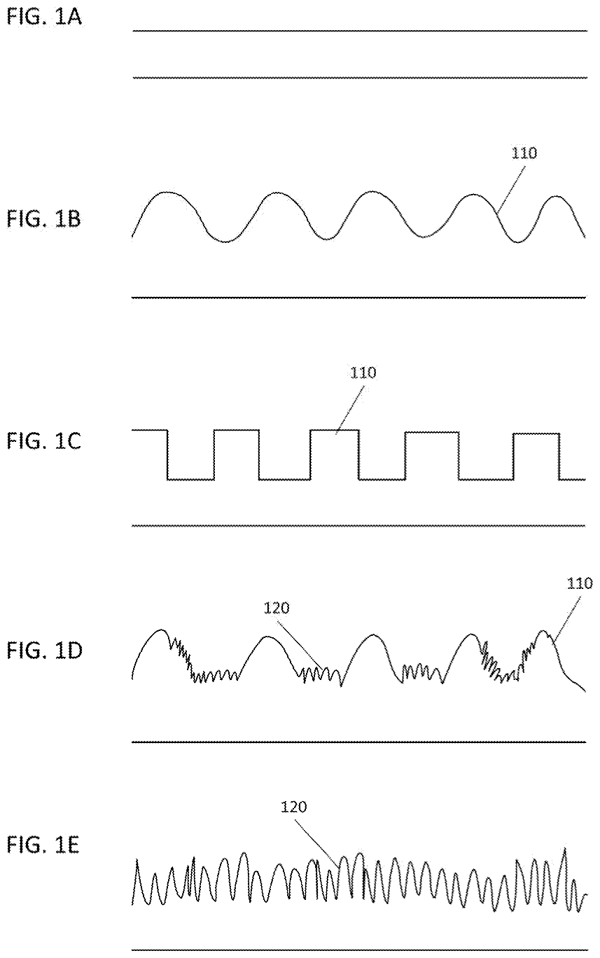

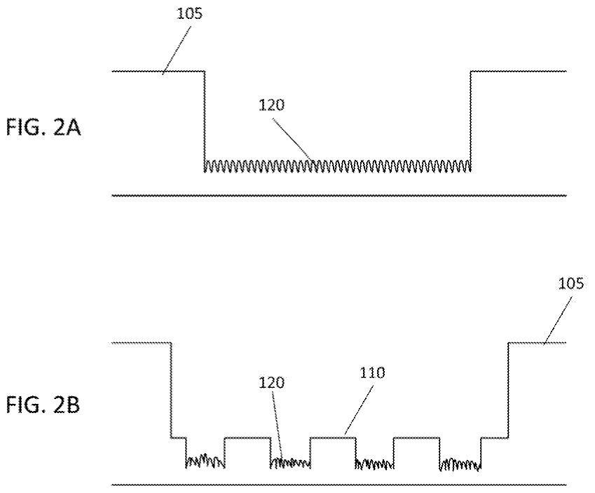

A textured surface or a textured substrate, as used herein, refers to a surface with elements, e.g., microfeatures and/or smaller microstructures, that increase the surface area as compared to a surface without microfeatures and/or microstructures (e.g., a flat, non-textured surface). In one embodiment, a system according to the present invention comprises a textured surface or substrate with elements (e.g., microstructures and/or microfeatures) that provide an increase in surface area as compared to a non-textured surface. The smaller microstructures may be disposed in or between (or both in and between, e.g., on sides of, etc.) the microfeatures (see FIG. 1D, FIG. 2B). A textured surface may comprise microfeatures without microstructures (see FIG. 1B, FIG. 1C). A textured surface may comprise microstructures without microfeatures (see FIG. 1E and FIG. 2A). The textured surface may or may not be part of a microarray substrate. Without wishing to limit the present invention to any theory or mechanism, it is believed that the use of a textured surface is advantageous because the textured surface has a substantial increase in surface area as compared to a flat, non-textured surface. The three dimensional nature of the textured surface can take many forms (as shown in FIG. 1B, FIG. 1C, FIG. 1D, FIG. 1E, FIG. 2A, and FIG. 2B). The microfeatures (and/or microstructures) may exhibit large values of height to width (aspect ratio). The high aspect ratio may be an indicator to performance of the structured surface, e.g., the higher the aspect ratio, the more area that is made available. U.S. Pat. No. 7,195,872 and EP No. 1,451,584, the disclosures of which are incorporated in their entirety herein by reference, describe examples of textured surfaces comprising a tessellating pattern of microfeatures, some of which comprise even smaller sized features (microstructures). The microfeatures and/or microstructures may comprise a pit, a trench, a pillar, a cone, a wall, a micro-rod, a tube, a channel, the like, or a combination thereof. The present invention is not limited to the patterning described therein.

Any feature or element (e.g., microfeature or microstructure) or combination of features or elements (e.g., microfeatures and/or microstructures) described herein is included within the scope of the present invention.

The microfeatures (or microstructures) may be distributed uniformly or randomly on the textured surface. The microfeatures may have a height from 0.1 μm to 100 μm, e.g., 100 μm, 90 μm, 80 μm, 70 μm, 60 μm, 50 μm. 40 μm, 30 μm, 20 μm, 10 μm, 1 μm, 0.5 μm, 0.1 μm, etc. In some embodiments, the microfeatures have a cross section (or an average cross section) from 0.01 μm² to 500 μm². The microfeatures may be at least 1 micron apart. In some embodiments, the microfeatures may be at least 5 microns apart. In some embodiments, the microfeatures are at least 10 microns apart. In some embodiments, the microfeatures are at least 15 microns apart. In some embodiments, the microfeatures are at least 20 microns apart. In some embodiments, the microfeatures are at least 50 microns apart. In some embodiments, the microfeatures are at least 100 microns apart. In some embodiments, the microfeatures are less than 500 microns apart. In some embodiments, the microfeatures are less than 100 microns apart. In some embodiments, the microfeatures are less than 50 microns apart. In some embodiments, the microfeatures are less than 20 microns apart. In some embodiments, the microfeatures are less than 10 microns apart. In some embodiments, the microfeatures are less than 5 microns apart.

As previously discussed, the microfeatures may exhibit large values of aspect ratio (height to width). Also, the aspect ratios of the various microfeatures may differ. In some embodiments, the microfeatures have an average aspect ratio of less than 25 (height divided by an average cross sectional width), or the microfeatures may each have an aspect ratio of less than 25. In some embodiments, the microfeatures have an average aspect ratio of less than 10 (height divided by an average cross sectional width), or the microfeatures may each have an aspect ratio of less than 10. In some embodiments, the microfeatures have an average aspect ratio of less than 5 (height divided by an average cross sectional width), or the microfeatures may each have an aspect ratio of less than 5. In some embodiments, the microfeatures have an average aspect ratio of less than 1 (height divided by an average cross sectional width), or the microfeatures may each have an aspect ratio of less than 1. The present invention is not limited to the aforementioned aspect ratio values.

Note the present invention is not limited to textured surfaces with microfeatures: in some embodiments the textured surface comprises only the smaller microstructures (e.g., see FIG. 1E, FIG. 2A). In some embodiments, the microstructures may be disposed in the microfeatures and/or between the microfeatures (e.g., see FIG. 1D, FIG. 2B). In some embodiments, the textured surface comprises only microfeatures (see FIG. 1B, FIG. 1C).

The microstructures may have a height less than 5 μm, e.g., 4 μm, 3 μm, 2 μm, 1 μm, etc. In some embodiments, the microstructures have a height less than 1 μm. In some embodiments, the microstructures have a height from 5 μm to 0.1 μm. In some embodiments, the microstructures have an average aspect ratio of less than 25 (height divided by an average cross sectional width), or the microstructures may each have an aspect ratio of less than 25. In some embodiments, the microstructures have an average aspect ratio of less than 10 (height divided by an average cross sectional width), or the microstructures may each have an aspect ratio of less than 10. In some embodiments, the microstructures have an average aspect ratio of less than 5 (height divided by an average cross sectional width), or the microstructures may each have an aspect ratio of less than 5. In some embodiments, the microstructures have an average aspect ratio of less than 1 (height divided by an average cross sectional width), or the microstructures may each have an aspect ratio of less than 1. The present invention is not limited to the aforementioned aspect ratio values.

The microstructures may be less than 500 nm apart. In some embodiments, the microstructures are less than 100 nm apart. In some embodiments, the microstructures are less than 50 nm apart. In some embodiments, the microstructures less than 20 nm apart. The microstructures may be at least 20 nm apart. The microstructures may be at least 10 nm apart. In some embodiments, the microstructures may be at least 50 nm apart. In some embodiments, the microstructures are at least 100 nm apart. In some embodiments, the microstructures are at least 500 nm apart.

The presence of the microfeatures may increase the surface area of the textured surface at least 10% as compared a surface without the microfeatures. The presence of the microfeatures may increase the surface area of the textured surface at least 50% as compared to a surface without the microfeatures. The presence of the microfeatures may increase the surface area of the textured surface at least 100% as compared to a surface without the microfeatures. The presence of the microfeatures may increase the surface area of the textured surface at least 200% as compared to a surface without the microfeatures. The presence of the microstructures may increase the surface area of the textured surface at least 10% as compared a surface without the microstructures. The presence of the microstructures may increase the surface area of the textured surface at least 50% as compared to a surface without the microstructures. The presence of the microstructures may increase the surface area of the textured surface at least 100% as compared to a surface without the microstructures. The presence of the microstructures may increase the surface area of the textured surface at least 200% as compared to a surface without the microstructures. The presence of the microfeatures and microstructures may increase the surface area of the textured surface at least 10% as compared a surface without the microfeatures and microstructures. The presence of the microfeatures and microstructures may increase the surface area of the textured surface at least 50% as compared to a surface without the microfeatures and microstructures. The presence of the microfeatures and microstructures may increase the surface area of the textured surface at least 100% as compared to a surface without the microfeatures and microstructures. The presence of the microfeatures and microstructures may increase the surface area of the textured surface at least 200% as compared to a surface without the microfeatures and microstructures.

The textured surface may be constructed from a material comprising a polymer material, a glass, a ceramic, a metal, the like, or a combination thereof. Polymers may include but are not limited to cyclic olefin copolymer (COC), polyethylene terephthalate (PET), polymethyl methacrylate (PMMA), polystyrene, the like, or a combination thereof.

The present invention features a system comprising a textured surface with elements and a non-volatile solvent. The textured surface with elements comprises microfeatures and/or microstructures that increase the surface area as compared to a flat surface (see FIG. 1A), e.g., a surface without microfeatures and/or microstructures. Biomolecules may be retained within or by the textured surface (e.g., microfeatures and/or microstructures) in the non-volatile solvent.

As previously discussed, the present invention, e.g., the textured surface featuring biomolecules in a non-volatile solvent (e.g., ionic liquid) retained within or by one or more microfeatures and/or microstructures of the textured surface, has an extended shelf life as compared to a flat surface with biomolecules anchored thereon because the biomolecules in the present invention are retained in an environment that is hydrated, that helps to preserve the conformational structure of the biomolecules, and that does not dry out. Thus, surfaces that incorporate the textured surfaces of the present invention allow for packaging, transporting, and storing of biomolecules adapted for assays such as microarrays or other applicable assays. In some embodiments, for example in an embodiment wherein the texture surface is part of a well, the system may feature a film that seals the opening of the well. Such a seal may help protect against any possible leakage or contamination from external sources. Generally, the surface energy of the ionic liquid would retain the mixture safely within the features of the textured surface.

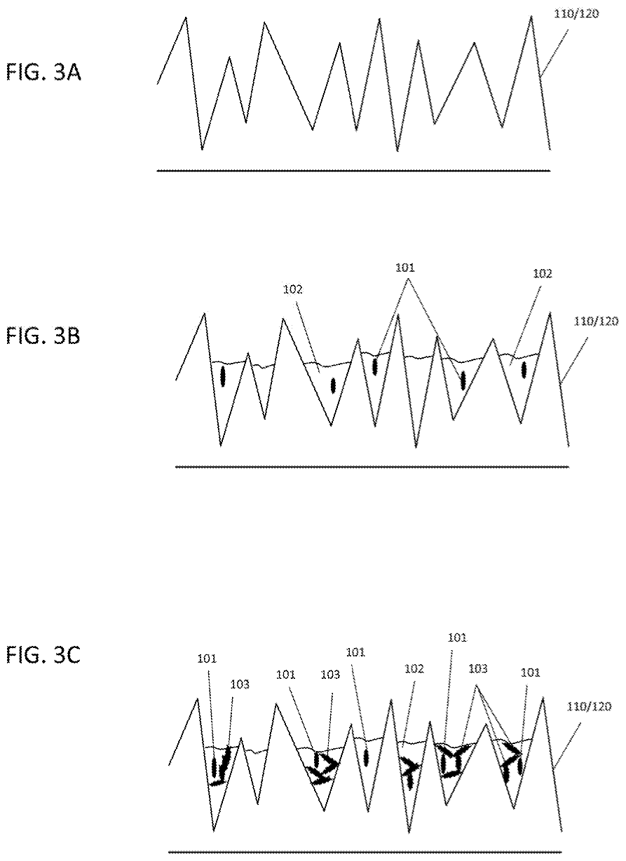

Note the textured surfaces of the present invention may or may not be part of a larger structure, such as a well or a microarray plate. For example, the present invention features textured surfaces such as those shown in FIG. 1B, FIG. 10, FIG. 1E, and FIG. 3A wherein a surface features one general type of texture (e.g., microfeatures (110) or smaller microstructures (120)). The present invention also features textured surfaces such as those shown in FIG. 1D wherein a surface features microfeatures (110) as well as smaller microstructures (120) within the microfeatures (110). The present invention also features textured surfaces such as those shown in FIG. 2A and FIG. 2B, wherein a surface with microfeatures (110) or a surface with both microfeatures (110) and microstructures (120) are part of a well. The present invention is not limited to microfeatures and/or microstructures as part of microarrays.

FIG. 3B shows an example of the present invention, e.g., a textured surface comprising microfeatures (110)/microstructures (120) and a non-volatile solvent (102), wherein biomolecules (101) are retained within or by the microfeatures (110)/microstructures (120) in the non-volatile solvent (102).

In some embodiments, the biomolecules are attached or anchored to the textured surface (e.g., microfeatures, microstructures). In some embodiments, the biomolecules are not attached or anchored to the textured surface (e.g., microfeatures, microstructures). For example, the confinement of the biomolecules (e.g., proteins or other biomolecules) within or by one or more of the microfeatures and/or microstructures may be sufficient to achieve both localization and retention. However, as previously discussed, the present invention also includes the use of attachment chemistries, which are known to one of ordinary skill in the art.

As previously discussed, the biomolecules may be retained within or by the microfeatures and/or microstructures of the textured surface. In some embodiments, the biomolecules are constrained from lateral movement between the different microfeatures and/or microstructures of the textured surface.

The present invention may also feature activating the textured surface by creating energetic moieties on the surface that allow for binding of a probe (e.g. a biomolecule), e.g., at least a portion of the textured surface (e.g., microstructures and/or microfeatures) comprise energetic moieties adapted to bind a biomolecule (e.g., an oligonucleotide, an amino acid, a peptide such as an antibody or fragment thereof, a carbohydrate, a lipid, or a combination thereof). The biomolecule may be bound to the microstructure directly via the energetic moiety. In some embodiments, a linker molecule is bound to the energetic moiety, and a biomolecule is bound to the linker molecule (e.g., a silane molecule). In some embodiments, the activated textured surface is a part of a surface of a slide, plate, microarray well, or a well of a multi-well plate.

Methods for activating the textured surface may comprise treating the textured surface with electromagnetic radiation (e.g., UV light) or plasma as described herein. For example, in some embodiments, the method comprises exposing a textured surface (e.g., with microstructures and/or microfeatures) to electromagnetic radiation (e.g., ultraviolet light) or plasma, wherein the electromagnetic radiation (e.g., ultraviolet light) or plasma activates surface molecules of the microstructures and/or microfeatures to yield energetic moieties adapted to bind biomolecules. However, the present invention is not limited to electromagnetic radiation (e.g., UV light) or plasma for surface activation/creation of energetic moieties. The present invention also includes any other appropriate surface activation method. The activated textured substrates of the present invention may result in improved sensitivity in diagnostic tests. In some embodiments, the methods of the present invention feature reducing the hydrophobicity of the textured surface to allow biomolecule binding.

The energetic moieties comprise a reactive species capable of binding biomolecules. In some embodiments, the reactive species comprises reactive oxygen species, a hydroxyl, an amine; a carboxyl, an aldehyde, an epoxy, or a combination thereof.

In some embodiments, the ultraviolet light is a wavelength of 400 nm or less, 270 nm or less, 185 nm or less, from 185 nm to 400 nm, etc. Doses may be from 25 to 1500 millijoules, e.g., 50 millijoules, 100 millijoules, 150 millijoules, 200 millijoules, from 25 to 100 millijoules, from 50 to 200 millijoules, from 100 to 500 millijoules, from 500 to 1500 millijoules, etc. In some embodiments, the ultraviolet light is used in combination with a gas (e.g., a gas comprising air, ozone, oxygen, nitrogen, nitrogen-hydrogen mixture, ammonia, argon, water vapor, or a combination thereof). In some embodiments, the plasma comprises atmospheric plasma or vacuum plasma.

Electromagnetic radiation treatment may feature exposure to a dose of UV light in the presence of a gas. The gas may comprise air, ozone, oxygen, nitrogen, nitrogen-hydrogen mixture, ammonia, an inert gas (e.g., argon), water vapor, the like, or a combination thereof. The selection of the gaseous material enables different species of energetic moieties to be created on the surface. Thus, the present invention allows for tailoring the binding surfaces (e.g., via gas selection) to match the nature of the incoming probe molecule (e.g. biomolecule). It will be appreciated that other means of creating these energetic moieties on the surface of the textured substrate are included in the scope of this invention. For example, the use of ozone may establish hydroxyl species on the surface; the use of nitrogen, nitrogen/hydrogen, or ammonia gases may establish amine species on the surface.

Plasma treatment may feature the use of atmospheric plasma or vacuum plasma. Without wishing to limit the present invention to any theory or mechanism, plasma may be chosen over ultraviolet light as it may be easier to use different gases to create tailored chemistries in the energetic moieties and the plasma process produces less heat in the textured substrate.

As an example, in some embodiments, the plasma treatment comprises exposing the textured substrate to plasma within a chamber (e.g., a chamber with electrodes adapted to generate a plasma environment). Plasma may include but is not limited to oxygen plasma, nitrogen plasma, plasma from oxygen and an inert gas (e.g., argon/oxygen plasma), nitrogen-hydrogen plasma, water vapor, or plasma from various other gases or combinations of gases.

The use of liquids that exhibit negligible vapor pressure as the liquid dissolving the biomolecules and retaining said liquids within the features of the textured surface helps to keep the biomolecules effectively hydrated.

Liquids that exhibit negligible vapor pressure (e.g., non-volatile solvents) and are solvents for biomolecules (e.g., proteins or other biomolecules) may include but are not limited to ionic liquids (e.g. protic), and one of ordinary skill in the art can recognize that the term “ionic liquids” includes the general class ionic liquids. The present invention is not limited to one or a few specific ionic liquids. Ionic liquids generally exhibit negligible vapor pressure over a wide range of conditions of temperature and pressure. It is known that proteins solvate well in ionic liquid salts. Protic ionic liquids can, in some conditions, become attached to the surface of the textured substrate by means of energetic moieties (e.g., as described above). This may help to enhance immobilization of the biomolecule (e.g., protein) without inflicting damage to the conformational structure of the biomolecule (e.g., protein). Additionally, these energetic moieties may also bind other polymerizable molecules used to help trap the biomolecule.

Liquids that exhibit negligible vapor pressure (e.g., non-volatile solvents) and are able to solvate biomolecules (e.g., proteins) include, but are not limited to, polyhydric alcohols, e.g., polyhydric alcohols that exhibit negligible vapor pressure at room temperature. In some embodiments, the efficacy of these liquids can be enhanced by the addition of saccharides. The proportion of hydroxyl groups may act beneficially to the process of protein solvation.

As used herein, biomolecules include but are not limited to proteins, oligonucleotides, lipids, carbohydrates, and the like. Non-limiting examples of proteins are enzymes and antibodies. In some embodiments, the biomolecule comprises a cell (e.g., a human cell or animal cell, a microbe, etc.), a virus, or other biological molecule.

The present invention also features the incorporation of polymerizable molecules (e.g., see FIG. 3B and FIG. 30). For example, a monomer of the polymerizable molecule (103) may be added to the liquid solution of the biomolecules. Subsequent polymerization of the polymerizable molecules into a gel-like environment may assist in the entrapment of the biomolecules (e.g., proteins) whilst remaining hydrated by the non-volatile liquid. Examples of polymerizable molecules include oligomers, acrylates, methacrylates, acrylamides the like, or a combination thereof. Polymerizable molecules are well known to one of ordinary skill in the art and are not limited to those disclosed herein.

The polymerizable molecules may be attached to an activated surface of the textured substrate, which may further enhance the immobilization of the biomolecules (e.g., proteins or other biomolecule). For example, an activated surface may refer to a surface featuring energetic moieties (e.g., as described above) that are able to capture a fraction of the ionic liquid molecules and a fraction of the polymerizable molecules. In some embodiments, anchoring polymerizable molecules to the textured surface may involve exposure to UV radiation. This both polymerizes the monomer and creates suitable linkages to the surface. The surface energy of a textured substrate can be modified by a variety of means to suit the particular purpose, e.g., exposure to various plasma environments, exposure to UV, selection of a suitable gas environment, etc.

As a non-limiting example, in some embodiments, a mixture comprising a protein dissolved in non-volatile, water-soluble solvent is delivered to the surface of the textured substrate and deposited at a specified location upon the surface of said textured substrate. The lateral movement of the droplet is limited by the lips of the well-like microfeatures. The droplet is absorbed into one or more adjacent well-like microfeatures. The droplet containing the protein molecule is further adsorbed into the smaller features within the well-like microfeature. A notable feature of the solvent is the property of having negligible vapor pressure and, as such, does not evaporate under most conditions of temperature and pressure. Thus the protein (or other bio molecule) has been deposited within the smaller elements of the well-like microfeatures and will remain well solvated because of the continued presence of the non-volatile solvent. The proteins are now accessible for any of a wide variety of diagnostic tests.

As a non-limiting example, in some embodiments, a mixture comprising a protein suspended in an ionic liquid is delivered to the surface of the textured substrate and deposited at a specified location upon the surface of said textured substrate. The lateral movement of the droplet is limited by the lips of the well-like microfeatures. The droplet is absorbed into one or more adjacent well-like microfeatures. The droplet containing the protein molecule is further adsorbed into the smaller microstructures within the well-like microfeature. Ionic liquids are selected because of their known compatibility with proteins. A notable feature of the ionic liquid is the property of having negligible vapor pressure. Ionic liquids do not evaporate under most conditions of temperature and pressure. Thus the protein (or other biomolecule) has been deposited within the smaller microstructures of the well-like microfeatures and will remain well solvated because of the continued presence of the ionic liquid. The proteins are now accessible for any of a wide variety of diagnostic tests.

As a non-limiting example, in some embodiments, a mixture comprising a protein suspended in a mixture of a non-volatile, water-soluble solvent and a small polymerizable monomer molecule is delivered to the surface of the textured substrate and deposited at a specified location upon the surface of said textured substrate. The lateral movement of the droplet is limited by the lips of the well-like microfeatures. The droplet is absorbed into one or more adjacent well-like microfeatures. The droplet containing the protein molecule is further adsorbed into the smaller microstructures within the well-like microfeature. A notable feature of the non-volatile, water-soluble solvent is the property of having negligible vapor pressure and, as such, does not evaporate under most conditions of temperature and pressure. Thus the protein (or other biomolecule) has been deposited within the smaller elements of the well-like features and will remain well solvated because of the continued presence of the non-volatile, water-soluble solvent. A second stage process involves the polymerization of the smaller monomer molecule. The polymer chains so produced create a gel-like environment that provides further containment for the protein. The proteins are now accessible for any of a wide variety of diagnostic tests.

As a non-limiting example, in some embodiments, a mixture comprising a protein suspended in a mixture of an ionic liquid and a small polymerizable monomer molecule is delivered to the surface of the textured substrate and deposited at a specified location upon the surface of said textured substrate. Prior to this deposition, the surface of the textured substrate is exposed to an activation process such that energetic moieties on its surface. These are designed to capture some of the monomer molecules and provide an initiation point for further polymerization. The lateral movement of the droplet is limited by the lips of the well-like features. The droplet is absorbed into one or more adjacent well-like features. The droplet containing the protein molecule is further adsorbed into the smaller features within the well-like feature. Ionic liquids are selected because of their known compatibility with proteins. A notable feature of the ionic liquid is the property of having negligible vapor pressure. Ionic liquids do not evaporate under most conditions of temperature and pressure. Thus the protein (or other biomolecule) has been deposited within the smaller elements of the well-like features and will remain well solvated because of the continued presence of the ionic liquid. The final stage process involves the polymerization of the smaller monomer molecule. The polymer chains so produced create a gel-like environment that provides further containment for the protein. In this embodiment, however, the polymerized chains are anchored to parts of the textured surface. Any energetic moieties residing within either microfeature of the microstructures may assist in the binding of the biomolecule to the textured surface. The proteins are now accessible for any of a wide variety of diagnostic tests.

Without wishing to limit the present invention to any theory or mechanism, it is believed that the availability of binding surface area in a textured surface may be beneficial for biomolecules that have to maintain their physical structure in order to participate in a diagnostic test. For example, biomolecules (e.g., proteins, e.g., antibodies) may be better able to survive in such textured surfaces like those of the present invention without denaturation and/or degradation. And, the methods, systems, and compositions of the present invention may allow for enhanced signals and/or better results when used for diagnostic or other purposes.

Any feature or element (e.g., microfeature or microstructure) or combination of features or elements (e.g., microfeatures and/or microstructures) described herein is included within the scope of the present invention.

The disclosures of the following patents are incorporated in their entirety by reference herein: U.S. Pat. App. No. 61/823,065; E.P. 1437368; E.P. 2233925; WO 2006062427.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

1. A system comprising: a textured surface with elements that provide an increase in surface area as compared to a non-textured surface, wherein a non-volatile solvent exhibiting negligible vapor pressure is contained within at least an element of the texture.

2. (canceled)

3. The system of claim 1 further comprising a biomolecule solvated in the non-volatile solvent.

4. The system of claim 3, wherein the biomolecule comprises an oligonucleotide, an amino acid, a protein, a carbohydrate, a lipid, or a combination thereof.

5. (canceled)

6. (canceled)

7. The system of claim 4, wherein the protein is an antibody or a fragment thereof.

8. (canceled)

9. (canceled)

10. The system of claim 1, wherein the non-volatile solvent comprises an ionic liquid salt.

11-14. (canceled)

15. The system of claim 1 further comprising at least one polymerizable molecule solvated in the non-volatile solvent.

16. The system of claim 1 further comprising cross-linked polymerized molecules solvated in the non-volatile solvent.

17-29. (canceled)

30. The system of claim 1, wherein the textured surface is a part of a surface of a slide, plate, microarray well, or a well of a multi-well plate.

31-33. (canceled)

34. The system of claim 1, wherein the system is used to retain and prevent denaturation of the protein molecule.

35. (canceled)

36. The system of claim 1 further comprising a layer overlaying the non-volatile solvent such that the non-volatile solvent is retained in the elements of the textured surface.

37-72. (canceled)

73. A method of preparing a system for retaining a biomolecule, said method comprising introducing a non-volatile solvent exhibiting negligible vapor pressure to a textured surface, the textured surface comprises elements that provide an increase in surface area as compared to a non-textured surface, wherein the biomolecule is solvated in the non-volatile solvent, wherein the non-volatile solvent with the biomolecule is adsorbed into the elements of the textured surface for retention of the biomolecule within or by the elements of the textured surface.

74. A method of storing a biomolecule, said method comprising: solvating said biomolecule in a non-volatile solvent and introducing said biomolecule to a system comprising a textured surface, the textured surface comprises elements that provide an increase in surface area as compared to a non-textured surface, wherein the non-volatile solvent with the biomolecule is adsorbed into the elements of the textured surface for retention of the biomolecule within or by the elements of the textured surface, wherein the biomolecule may be stored for a period of time.

75. (canceled)

76. (canceled)

77. The method of claim 74, wherein the system further protects a conformational structure of the biomolecule.

78. The method of claim 74 further comprising introducing to the textured surface a polymerizable monomer molecule, the polymerizable monomer molecule is adsorbed into the elements of the textured surface with the non-volatile solvent and biomolecule.

79. (canceled)

80. (canceled)

81. The method of claim 78, wherein polymerizing the polymerizable molecule comprises exposing said polymerizable molecule to an external stimulus, and wherein the external stimulus is electromagnetic radiation.

82. (canceled)

83. (canceled)

84. The method of claim 74 further comprising exposing the textured surface to electromagnetic radiation or plasma to generate energetic moieties displayed on the elements of the textured surface, the energetic moieties are adapted to attach the biomolecule to the textured surface, and wherein the plasma comprises atmospheric plasma or vacuum plasma.

85-92. (canceled)

93. The method of claim 74, wherein the biomolecule comprises an oligonucleotide, an amino acid, a protein, a carbohydrate, a lipid, or a combination thereof.

94. The method of claim 74, wherein the biomolecule comprises a cell, virus or bacteria.

95-112. (canceled)

113. The method of claim 74 further comprising a layer overlaying the non-volatile solvent such that the non-volatile solvent is retained in the elements of the textured surface.

114-116. (canceled)

117. The method of claim 74, wherein the of time is at least 1 month.

118. (canceled)
